(19) United States
(12) Patent Application Publication
(10) Pub. No.: US 2008/0221688 A1
(43) Pub. Date: Sep. 11, 2008

(54) Method of Maintaining Fatigue Performance In A Bone-Engaging Implant

(75) Inventors: Hai H. Trieu, Cordova, TN (US); Frank Bono, Collierville, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/684,135

(22) Filed: Mar. 9, 2007

(51) Int. Cl. A61F 2/44; A61B 17/58

(52) U.S. Cl. 623/17.16; 72/362

(57) ABSTRACT

This disclosure is directed to a method capable of maintaining the fatigue performance of a bone-engaging implant after surface texturing through the imparting of a residual compressive stress. A residual compressive stress is imparted via peening or another process. The peened surface is then roughened to improve mechanical adhesion for bone fixation via grit blasting or another process. The depth of penetration of the roughened texture is less than the depth of the residual compressive stress.

### FIELD OF THE INVENTION

The present invention relates generally to the field of preparing medical devices for implantation.

### BACKGROUND

Some implants include roughened bone-engaging surfaces that increase friction between implant surfaces and bone structures. While potentially beneficial for maintaining the implant in its desired location relative to the mating bone structure, some surface roughening procedures also may introduce microscopic stress risers. Further, in some static and some dynamic applications, the implant may be subject to loading, including cyclic loading. Over time as a result of the loading, the implant may fatigue, and cracks may initiate at the stress risers. Further loading may exceed the limits of the device, and propagate cracks that lead to failure of the device. Increasing resistance to fatigue may extend the recommended life cycle of the implant.

The present disclosure is directed to a method of maintaining fatigue performance of an implant to overcome one or more deficiencies in the art.

### SUMMARY

In one exemplary aspect, the present disclosure is directed to a method including introducing a residual compressive stress into a bone-engaging portion of an implantable device configured for implantation in a body. The bone engaging portion may include a bone-engaging surface. The method also may include texturing the bone-engaging surface of the implantable device to increase a roughness of the bone-engaging surface.

In an exemplary aspect, the introducing a residual compressive stress is performed until the compressive stress is at a first depth in the bone engaging portion, and wherein texturing the bone-engaging surface is performed until the texturing is at a second depth in the implantable device, and wherein the second depth is less than the first depth.

In an exemplary aspect, the introducing a residual compressive stress includes shot peening the bone-engaging portion and the texturing the bone-engaging surface includes grit blasting.

In another exemplary aspect, the present disclosure is directed to a method of treating a bone-engaging portion of an implantable device to maintain fatigue resistance properties while providing surface texturing. The method may include peening the bone-engaging portion of the implantable device to introduce a residual compressive stress into the bone engaging feature. The residual compressive stress may have a first depth. The method also may include texturing the peened bone engaging portion to increase a surface roughness of the bone engaging portion. The texturing may have a second depth.

In an exemplary aspect, the second depth is less than the first depth.

In an exemplary aspect, the texturing comprises grit blasting the bone-engaging portion of the implantable device.

In another exemplary aspect, the present disclosure is directed to an implantable device. The device may include a bone-engaging portion having a bone-engaging surface and a thickness. The bone-engaging portion may have a residual compressive stress extending to a first depth. The bone-engaging surface also may have a roughened texture. The roughened texture may penetrate the bone engaging surface to a second depth that may be less than the first depth.

Further aspects, forms, embodiments, objects, features, benefits, and advantages of the present invention shall become apparent from the detailed drawings and descriptions provided herein.

### BRIEF DESCRIPTION OF THE DRAWINGS

FIG. 1 is an exemplary embodiment of a vertebral member having two bone-engaging implants.

FIGS. 2A-2D are illustrations of a cross sectional view of an exemplary portion of the implant shown in FIG. 1. FIG. 2D also includes a stress graph showing stress in the exemplary portion of the implant.

FIGS. 3A-3C are illustrations of a cross sectional view of the exemplary portion of the implant shown in FIGS. 2A-2D. FIG. 3C also includes a stress graph showing stress in the exemplary portion of the implant.

FIG. 4 shows a cross section of an exemplary portion of the implant shown in FIG. 3C.

FIGS. 5-10 are illustrations of exemplary embodiments of implantable devices treated according to the process disclosed herein.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The systems, devices, and methods described herein may be used to increase the projected useful life of bone-engaging implants. Some conventional implants include bone-engaging surfaces which have been treated to introduce surface irregularities, or treated to increase surface roughness. Increased surface roughness cooperates with adjacent bone to frictionally secure the implant in place. In addition, an implant may biomechanically engage an adjacent bone as the bone grows into irregularities and imperfections available on a roughened surface.

A roughened surface, although advantageous for securing an implant, may be detrimental to an implant's fatigue strength. Forces or loads repeatedly introduced during shifting of weight, such as during patient movement, impart cyclic stress on the implant. This cyclic stress presented over time may fatigue the implant, lowering its estimated useful life.

Stress typically concentrates at certain locations, such as at surfaces and at certain physical features. The physical features where stress concentrates may be intentional, such as at a sharp corner, or unintentional, such as at a crack. A microscopic, or near microscopic crack in the surface of a load bearing object may present a potential stress concentration as a stress riser. Cyclical loading of a surface impaired with such a stress riser may lead to a shortening of the product's useful life.

For example, in a conventional material, a tensile stress concentrated at a crack tends to pull the crack open. As the crack opens, the root of the crack travels deeper into the surface, thereby further reducing a cross-section of the material available to resist the tensile load. Thus, even more stress is concentrated into the crack. Each time the tensile load is applied, the crack deepens, and if unabated, the repetitive, or cyclic loading will drive the crack deeper until the load bearing object is rendered unusable by such fatigue cracking.

Increasing resistance to effects of cyclic loading, for example, as against tensile forces acting on the surface, may increase an implant's projected life. More particularly, using the systems, devices, and methods disclosed herein, increased resistance to fatigue failure may be achieved while still maintaining the implant's ability to cooperate with the bone to frictionally or biomechanically engage the bone.

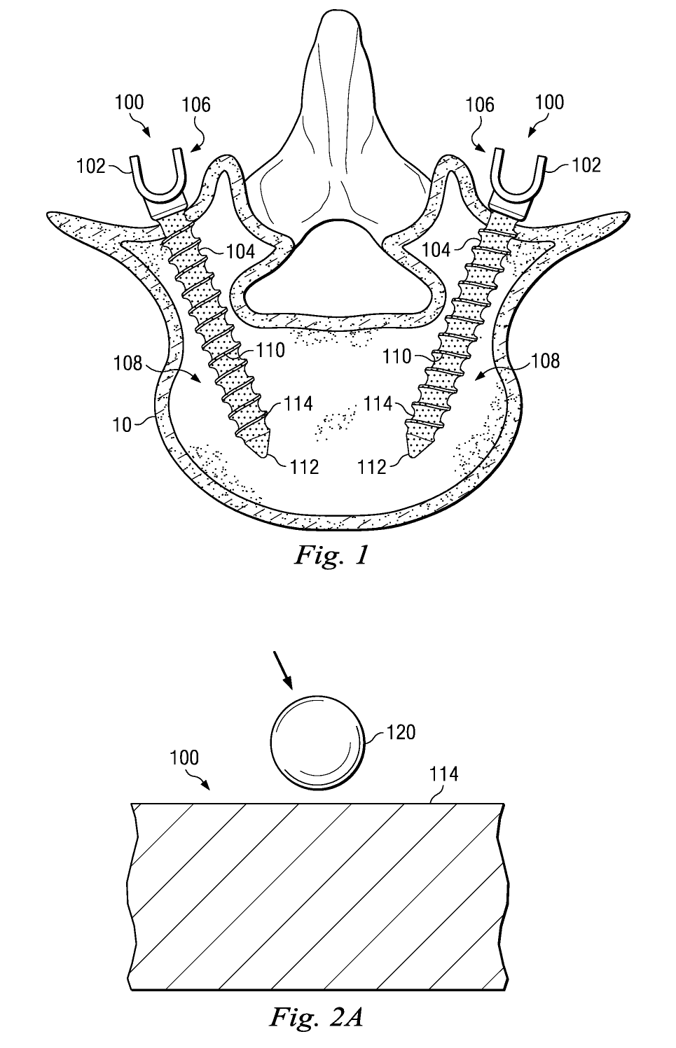

Turning now to FIG. 1, an exemplary embodiment of a vertebral member 10 is illustrated having two bone-engaging implants, each generally referenced by the numeral 100, mounted within. In this embodiment, each of the implants 100 is substantially the same although it is understood that embodiments with multiple implants 100 may include different features or specifications. Each implant 100 includes a head 102 and a shaft 104. The head 102 is disposed at a proximal end 106 of the implant 100 and the shaft 104 forms a distal end 108. The shaft 104 includes radially extending threads 110 spiraling from the head portion to a tip 112 at a distal most end. In this exemplary embodiment, the shaft 104 includes a bone-engaging outer surface 114 configured to interface directly with the bone tissue of the vertebral member 10.

In order to increase the estimated useful life of the implant, the bone-engaging surface 114 may be treated to maintain fatigue resistance properties while promoting bone integration and frictional resistance to displacement. In this example, the bone engaging surface 114 may be treated to first introduce compressive stress to the implantable device, followed by a treatment to roughen or texture the bone-engaging surface of the implant.

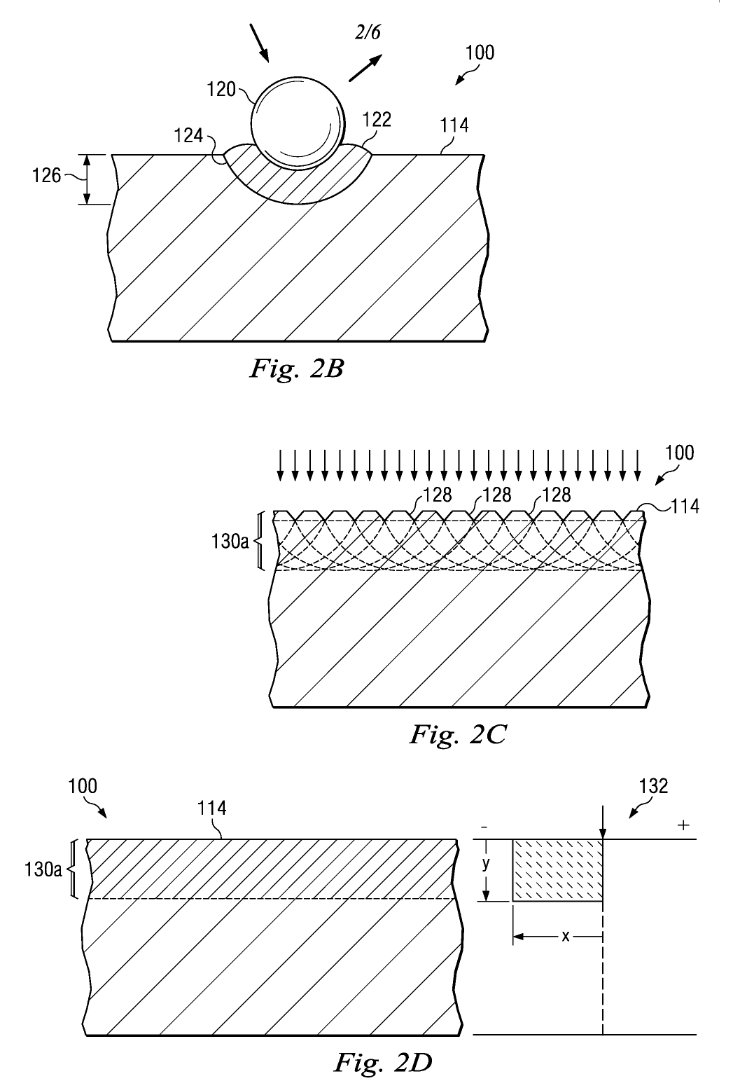

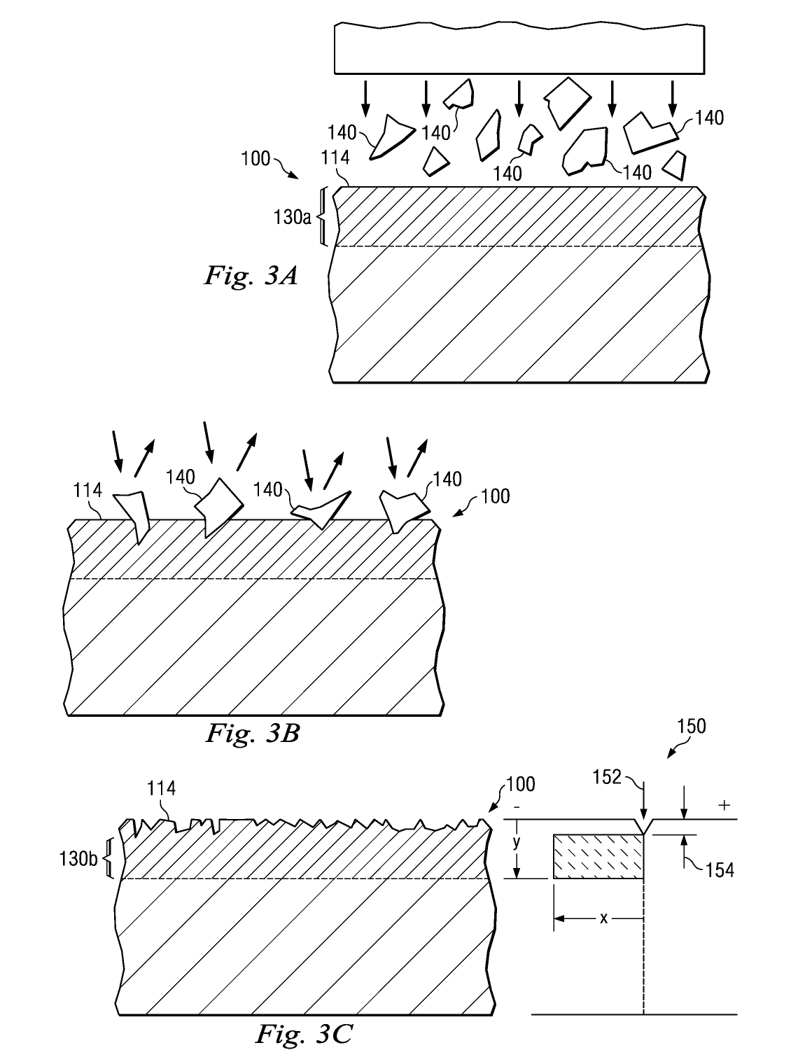

One exemplary process for treating the bone-engaging surface 114 is described with reference to FIGS. 2A-2D and FIGS. 3A-3C. In this example, the bone engaging surface 114 of the implant is work-hardened, and in this case cold-hardened, by shot peening the surface 114. The process for doing this, including its effects, are described below with reference to FIGS. 2A-2D. Following the work-hardening, an exemplary surface texturing process, grit blasting in this example, is described with reference to FIGS. 3A-3C.

The surface of the implant 100 may be described as being comprised of many layers of atoms arranged in a lattice, or matrix. Spaces or voids, as well as out-of-place metal atoms or interstitials, are interspersed throughout the matrix. It is possible to force interstitials, along with otherwise aligned atoms, into these voids in a deeper surface layer. Plastic deformation occurs during the permanent dislocation of a metal atom, resulting in a breaking of existing atomic bonds followed by subsequent re-bonding in a new location. If a dent is limited to a dimple on only one side of a work piece, the atoms have been compressed into a smaller space. The area of the plastic deformation contains more atoms, hence more electrical bonds. As more and more atoms occupy the same space in a metal, that space's ability to deform plastically diminishes so that working more metal atoms in the same space creates a stronger metal, albeit with less ductility.

Thus, the compression of metal atoms en mass both hardens and strengthens the material. This strengthening of the metal is termed strain-hardening, or work-hardening, and it is accomplished through plastic deformation. In addition, work-hardening results in a residual compressive stress. Essentially, any applied tensile forces may be countered by the compressive force already existing in the surface layers. Shot peening is one method of work-hardening a material, such as the surface 114 of the implantable device 100.

Referring now to FIGS. 2A-2D, FIG. 2A shows a cross section of an exemplary portion of the implant 100, with the outer bone-engaging surface 114 of the shaft 104. A single shot 120, is represented as a spherical or round ball and is shown traveling towards the bone-engaging surface 114. At impact, as shown in FIG. 2B, a large amount of kinetic energy is transferred from the single shot 120 to the bone-engaging surface 114. However, the single shot 120 maintains a portion of its kinetic energy enabling it to rebound away from the bone-engaging surface 114.

Although some energy is dissipated as heat and other energy potentially lost through break-up of a shot particle, the remaining energy is transferred into the bone-engaging surface 114. The instantaneous transference of energy upon impact physically displaces a volume of metal at the point of impact, as shown in FIG. 2B. An impact of sufficient intensity plastically deforms the displaced metal, leaving a dimple after the single shot 120 travels away. A lesser amount of energy might only elastically deform the displaced metal, thereby leaving a surface mechanically unaffected.

Upon impact, an unconstrained portion of the displaced metal plastically deforms into free space on either side of the single shot 120, forming ridges 122 with a raised, rounded edge.

A constrained portion 124 of the area around the impact is bounded and unable to plastically deform into free space. The constrained portion 124 is work-hardened as a certain volume of metal atoms is compressed into a lesser volume. The work-hardened or constrained portion 124 has a thickness or depth 126 that corresponds to the amount of energy imparted upon impact of the single shot 120.

FIG. 2C illustrates the results of further peening of the bone-engaging surface 114. Dimples 128 created by peening begin to overlap, resulting in a uniform compressive layer 130a at the surface of the bone-engaging surface 114. The compressive layer 130a squeezes the grain boundaries of the bone-engaging surface material together, creating a layer of crack-resistant material. Thus, the ability of the implant to resist fatigue cracking is increased.

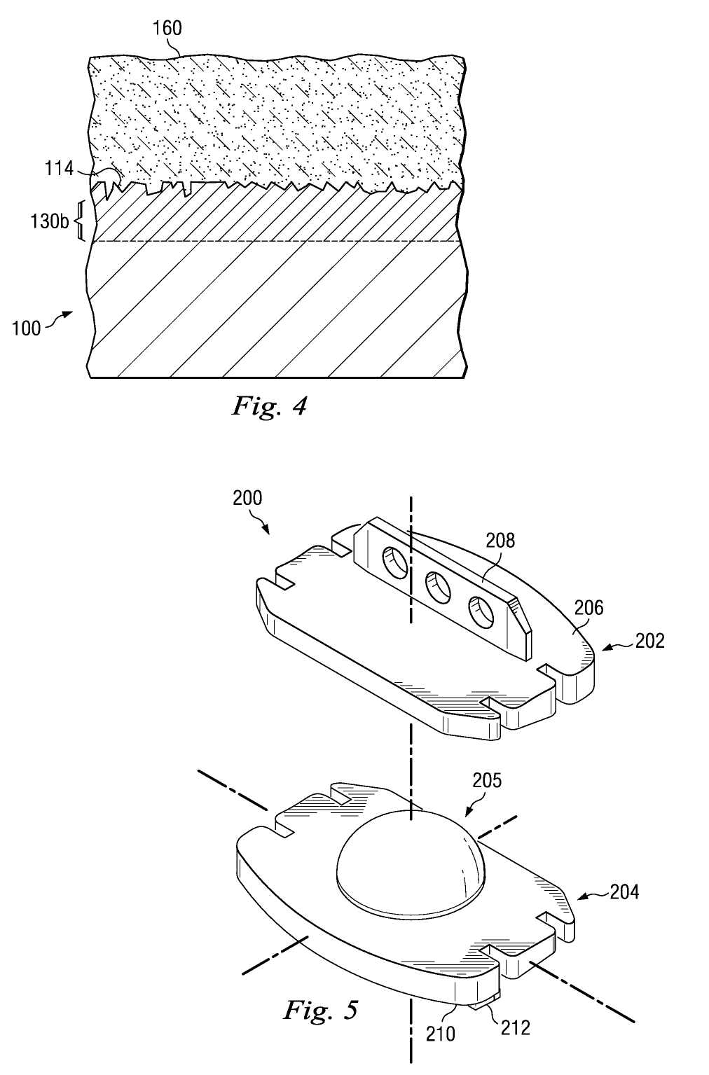

FIG. 2D shows the surface 114 having the compressive layer described in FIG. 2C after additional peening, where the high points are eventually compacted down, leaving a dimpled surface (not shown). In addition, FIG. 2D shows a stress graph 132 representing the corresponding stresses and their magnitudes of the implant 100 in FIG. 4D. The stress graph 132 is a simplified graphical representation of the stress experienced at a point as the stress travels down through the surface 114. On the stress graph 132, the negative symbol represents compressive stress while the positive symbol represents tensile stress. A residual compressive stress, due to the peening, is shown by this graph 132. The horizontal distance x away from the vertical axis represents the relative magnitude of the compressive stress at vertical depth y. The stress graph 132 is bounded horizontally by an upper surface and a lower surface of the implant. Beyond vertical depth y, the residual compressive stress is nominal. The vertical depth y shows how deep the compressive stress extends into the surface 114.

The depth of the compressive layer in shot peening is dependent on a number of controllable factors, including shot size, shot material, shot velocity, distance between the surface and the nozzle, angle of impact and time under shot peen. Other considerations include the repair status of the shot peen device, the degradation of the shot peen media over time and the internal degradation of the shot peen device over time.

While shot peening in general can change the appearance of a surface, only the deeper, plastically-deforming dimples result in improved mechanical properties. Therefore, it is useful to be able to determine the depth and consistency of coverage.

Generally, there are two measurements used to verify the shot peening process. “Coverage” refers to the degree of overlap of dimples that is attained. Coverage can be examined visually and directly. “Intensity” refers indirectly to the amount of plastic deformation imparted to the target material.

However, the intensity and consistency of coverage cannot be directly equated to desired mechanical conditions without resorting to destructive test methods. Non-destructive test methods such as X-ray radiography, mag-particle inspection, ultrasonic testing, visual inspection, dye penetrant inspection, eddy current testing, and coupon testing and correlation, among others, can be used as indirect measures of depth and consistency.

One method of verifying coverage and intensity employs Almen strips. These uniform steel test coupons physically deform under peening, indicating the coverage and intensity. These may be used in test experiments that subject the same implantable device to increasing amounts of peening time. Other factors are held constant throughout the experiment such as shot velocity, location of the implant, shot size and quality, angle of impact, material and shape of the implant and Almen strip manufacturing lot, among others. Subsequent to, or in conjunction with the shot peening of the sample implant, an Almen strip is shot peened under the same controlled conditions. The peening time is then increased, and the test is repeated. As residual compressive stresses accumulate, the Almen strip test coupon begins to curve. At each setting or parameter, the curvature of the Almen strip is measured and the corresponding implant is destructively tested by metallographic sampling. When the metallurgical sample exhibits the desired depth and consistency of shot peening, the curvature of the corresponding Almen strip will be measured and charted. The correlation between Almen strip curvature and actual surface compression produces a reliable and repeatable verification method. Hence, the shot peen process can then be manipulated as desired while ensuring that the process imparts a consistent depth of compression to the shot-peened surface.

Various implant features and base materials require varying process controls to obtain a sufficient compressive depth. One variable is the type and geometry of the shot media, which must not have an adverse effect on the target material's metallurgy or surface strength. The media, or shot may be made from cast steel, conditioned cut wire steel, glass, and ceramic, among other materials. The shape of shot may be approximately round as in the case of conditioned cut wire, or actually spherical as in the case of ball bearings.

One experienced in the art of shot peening will be familiar with other variations in establishing the correlation data for verification of the process and the best parameters and machinery to use for a particular implant. In some applications an implant may require partial masking to protect sensitive portions.

The resulting surface after shot peening may include small rounded ridges and dimples. In order to further improve the surface 114 to promote even additional surface bone-engaging texturing, the surface 114 in some embodiments, may be exposed to additional processing. This additional processing may create a surface that even more fully promotes bone integration and frictional resistance to displacement.

A processing step following shot peening applies a further more random roughening of the surface. In this embodiment, the bone-engaging surface 114 is textured, or roughened, beyond what is attainable through shot peening alone.

FIG. 3A-3C show an exemplary process for roughening or texturing the surface of the implant 100.

Turning to FIG. 3A, the work-hardened bone-engaging surface 114, with its compressive layer 130a, is subjected to an additional texturing treatment. In this embodiment, the texturing is accomplished by grit blasting. This includes pneumatically hurling grit particles 140 at a high velocity at the bone-engaging surface 114. Unlike the shot peen media described above, the grit particles 140 contain edges, corners, and non-uniform sizes and shapes. FIG. 3B shows some of the grit particles 140 engaging the bone engaging surface 114 of the implant 100. Corners and edges of the grit particles create small impressions, gouges, and the like in the bone-engaging surface 114 by plastic deformation or material removal, thereby roughening the surface. This both increases the overall surface area of the upper surface and increases the capacity of the bone-engaging surface 114 to mechanically interlock with bone tissue as the impressions and gouges receive boney ingrowth.

The grit blasting process may include any known grit, which is selected based on a survey of the target material used for an implant. Grit particles may be formed of glass, sand, metal, polymers, slag, alumina oxide, among others. Typically, though not always, the selected grit particles are harder than the implant material.

Grit blasting alone, while useful for improving coating adhesion, can create stress risers leading to a shortened useful life. FIG. 3C shows the bone-engaging surface 114 after the texturing process in conjunction with a corresponding stress graph 150. As can be seen, the bone engaging surface 114 includes irregularities such as notches and nicks that increase the surface roughness of the implant 100. These irregularities reduce some of the residual compressive stress introduced during the shot peening process; however, the irregularities do not fully penetrate the compressive layer 130a. This phenomenon is further illustrated by the stress graph 150. In this simplified graphical representation of the stress experienced in the surface 114, an exemplary notch 152, representing a notch in the surface 114, is shown in the graph 150. As can be seen, the notch 152 represents a removed portion of the surface 114, resulting in a decrease of the residual compressive stress down to a notch depth 154. Hence, the total compressive stress shown in the graph 150 of FIG. 2C has been reduced by the difference between the notch depth 154 and the vertical distance y shown in FIG. 3C. In effect, a tensile stress at the surface 114 is met with less counter-acting compressive residual stress. However, as can be seen, a relative amount of residual compressive strength 130b remains at the notch point 152, providing resistance to crack propagation. This benefit will continue to inure as long as the notch depth 154 is less than the vertical depth y of the residual compressive stress. In some exemplary embodiments, the roughening process is established to roughen the surface 114 to a depth that is less than about 50% of the depth of the compressive layer. Other depths, both greater and smaller also are contemplated.

Thus, as shown in FIG. 3C, by carefully controlling the shot peening and grit blasting processes, a residual compressive stress benefit can be combined with a surface roughening benefit.

Returning to FIG. 1, the shaft portion 104 of the implant 100, including the bone engaging surface 114 is work-hardened and then textured. However, in other exemplary embodiments, only a portion of the bone-engaging surface 114 is work-hardened and textured. For example, in some exemplary embodiments, the entire implant 100 is work-hardened, but only the bone-engaging surface 114 is textured. In other exemplary embodiments, only a part of the bone engaging surface 114 is textured. In yet others, only a part is work-hardened. Other combinations also are contemplated.

FIG. 4 shows one example of the implant 100 implanted in bone 160 adjacent the bone engaging surface 114. Over time, the bone tissue 160 grows into the cracks, scores, and markings on the bone engaging surface 114, mechanically securing or mechanically interlocking the bone 160 to the bone engaging surface 114, restricting removal of the implant 100. In addition, the compressive layer 130b continues to inhibit crack propagation at the bone engaging surface, thereby prolonging the estimated useful life of the implant 100.

Although the above example uses shot peening for the work-hardening process, other work-hardening processes also may impart a suitable compressive layer to the implant. For example, in some exemplary processes, the compressive stress layer is introduced to the implant using a forging process, a pressurization process, a water jet process, a drawing process among other processes and treatments. Cold-working treatments may be used to work harden the implant 100. Some of these may include, for example, cold rolling, roll forming, drawing, deep drawing, pressing, bending, cold forging, cold extrusion, hammering, and shearing, among others.

Alternatively, other forms of work-hardening via peening processes other than shot peening can be used. For example, laser peening uses shock waves to induce residual compressive stress. This may be useful when a very deep, or tightly controlled compressive layer is desired. Strain peening also may be used, whereby the implant is pre-strained below its elastic limit so that the bone-engaging surface is in tension is followed by shot or laser peening the surface to create a compressive layer, and then releasing the implant to impart further compression as it returns to its original form. Dual peening may be used to introduce additional compression by shot peening a second time with a smaller-sized shot.

Also, it is noted that grit blasting is just one example of a texturing process that may be used to promote bone integration and frictional resistance to displacement. Other suitable processes include, for example, chemical or electrical etching, sanding, electrical discharge, grit-blasting, abrading, plasma etching, or embedding particles within the surface. Yet other processes for texturing or roughening the surface of the implant 100 are contemplated.

Also, it should be noted that treatment of the entire bone contacting surface or a portion of the bone contacting surface may be suitable to impart the strength and surface texture desired. For example, in FIG. 1, in some exemplary embodiments, only the distal end portion near the tip may be treated with the roughening process while the entire bone engaging shaft 104 may be treated with the work-hardening process. Yet other arrangements are contemplated. In some examples, the bone engaging surface is treated in a pattern or spot treated to achieve desired properties and a desired interface.

FIGS. 5-11 shown some examples of additional implants that may be treated to increase their life expectancy. Referring first to FIG. 5, an exemplary implant, referenced herein by the reference numeral 200 is a motion preserving spinal disc configured for implantation between adjacent vertebrae to replace a natural spinal disc. The implant 200 includes an upper portion 202 and a lower portion 204 having features that together form a ball and socket type articulating joint 205 that provides relative rotation between the adjacent vertebrae.

The upper portion 202 includes an upper surface 206 and a keel 208, while the lower surface includes a lower surface 210 and a keel 212. These surfaces 206, 210, along with surfaces of the keels 208, 212 act as bone engaging surfaces that interface with the bone tissue of the adjacent vertebrae. The bone engaging surfaces may be treated by a work-hardening process to increase fatigue resistance and then by a texturing process to increase the capacity of the bone engaging surfaces to mechanically engage adjacent bone tissue. In some embodiments, only the upper and lower surfaces are treated, while in other embodiments only the keels are treated. In yet other embodiments, the keels and the outer surfaces are treated. Some embodiments may include only a portion of a surface to be treated with one or both of the blasting and texturing processes.

In some embodiments, the ball and socket joint components may be highly polished and any imperfections may be undesirable. Therefore, a manufacturer may desire to protect the ball and socket joint 205 from either the ball peening or from the grit blasting material. Accordingly, prior to the ball peening and blasting processes, the ball and socket joint components may be masked so as to protect them from accidental peening and blasting.

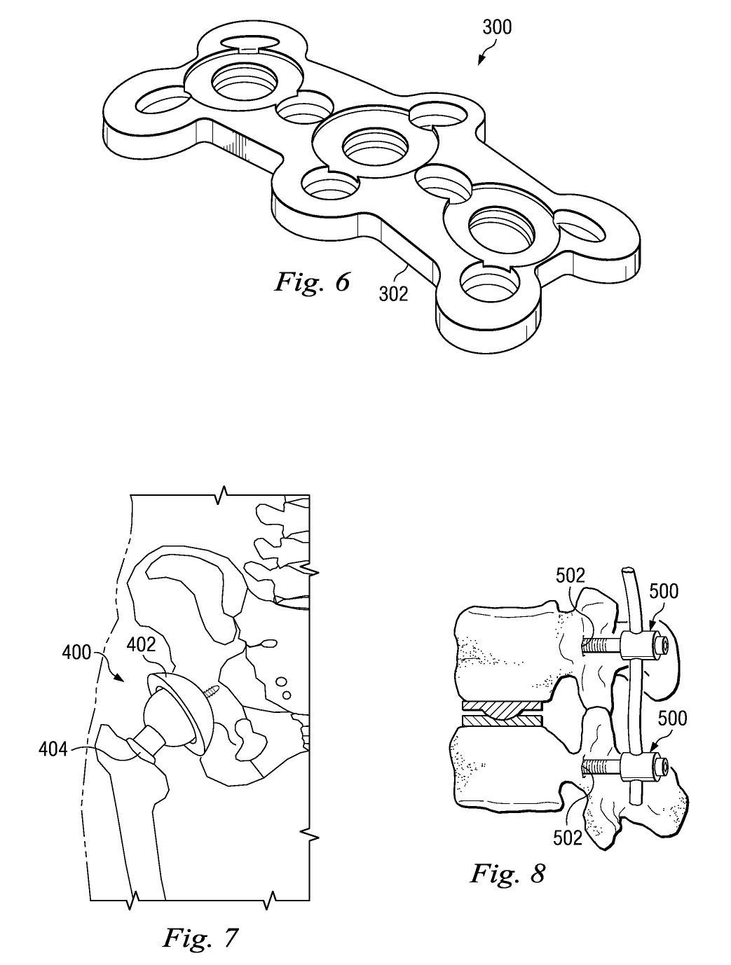

FIG. 6 shows another exemplary embodiment of an implant, referenced herein by the numeral 300, that may be treated. In this exemplary embodiment, the implant is a bone plate that may span an intervertebral disc space and attach to adjacent vertebrae using implantable bone anchors. The implant includes bone engaging surfaces 302 that may be resistant to fatigue and may include texturing. The implant may include a lower surface that may be a bone engaging surface and may be treated to reduce fatigue and include proper surfacing.

FIG. 7 is yet another exemplary embodiment of an implant, referenced herein by the numeral 400. In this exemplary embodiment, the implant is an implantable prosthetic hip joint having an outer bone engaging surface 402 and a bone engaging hip stem 404. The implant 400 includes bone engaging surfaces that may be resistant to fatigue and may include texturing. The bone engaging surface 402 and bone engaging hip stem 404 may be treated through a work-hardening process and a texturing process as described above to provide the desired qualities and characteristics.

FIG. 8 is yet another exemplary embodiment of an implant, referenced herein by the numeral 500. In this exemplary embodiment, the implant is, as in FIG. 1, a bone anchor. Here the bone anchor is in the form of a pedicle screw. A portion of the bone anchor may protrude into a part of the vertebra, such that a bone engaging outer surface 502 interfaces with the vertebra. The bone engaging surface 502 may be treated through a work-hardening process and a texturing process as described above to provide the desired qualities and characteristics.

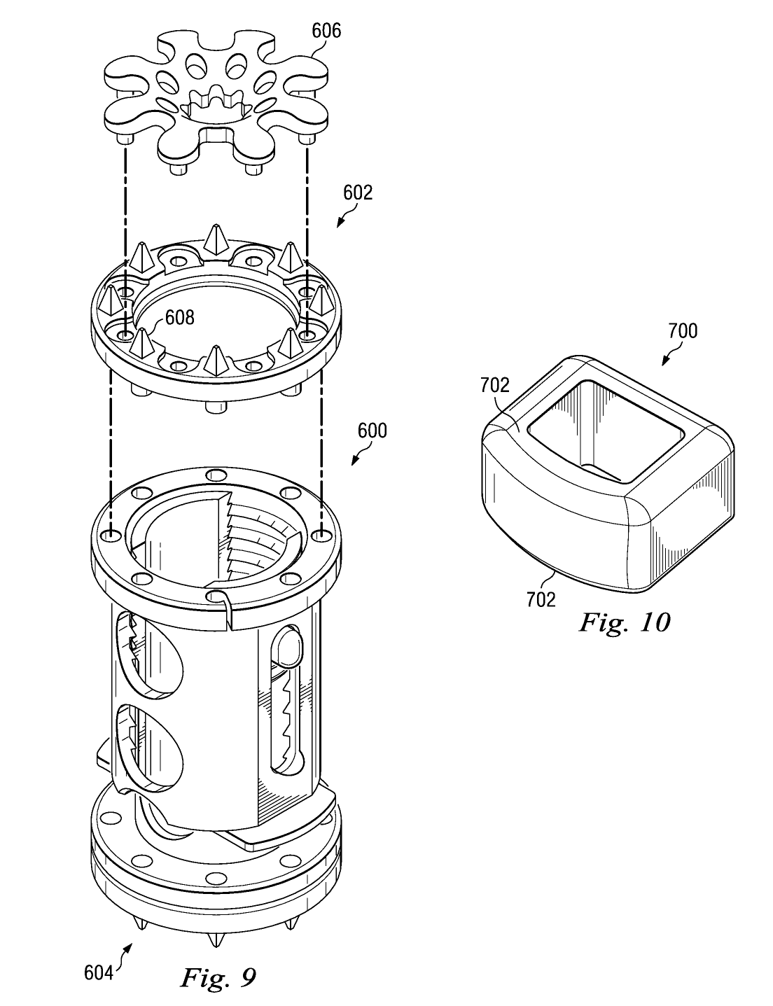

FIG. 9 is yet another exemplary embodiment of an implant, referenced herein by the numeral 600. In this exemplary embodiment, the implant is a corectomy device configured to replace a vertebral body. Ends 602, 604 may include bone engaging features, such as the basket 606, spikes 608, or other bone engaging surfaces. All or a part of one or more of these surfaces may be treated through a work-hardening process and a texturing process as described above to provide the desired qualities and characteristics.

FIG. 10 is yet another exemplary embodiment of an implant, referenced herein by the numeral 700. In this exemplary embodiment, the implant is an intervertebral spacer configured to fit within an intervertebral space between adjacent vertebrae. The spacer includes bone engaging surfaces 702 that may interface with the upper or lower vertebra. All or a part of one or more of these surfaces may be treated through a work-hardening process and a texturing process as described above to provide the desired qualities and characteristics.

FIGS. 5-10 show a few examples of implants finding utility for the process of maintaining fatigue performance described herein. Yet other implants may be treated by the disclosed processes and include the disclosed features. Some examples of other suitable implants include a disc replacement device, a facet joint replacement implant, an interspinous spacer, a bone screw, a bone anchor, a bone fastener, a fenestrated screw, a corpectomy device, an intramedulary rod, a hip joint replacement implant, a bone pin or rod, a knee joint replacement implant, a shoulder joint replacement implant, an elbow joint replacement implant, a wrist joint replacement implant, an ankle joint replacement implant, a finger joint replacement implant, a toe joint replacement implant, a dental implant, and a maxillofacial/cranial implant. These are just example, and others also are contemplated.

The implants need not be under cyclic load to benefit from the process disclosed herein. Accordingly, any bone engaging surface may be benefited from the processes disclosed herein. For example, in addition to the implants mentioned herein, the process may be used to increase the fatigue resistance and the bone engaging properties of implantable devices, such as bone pins and bone screws. As described above, these processes may find particular utility when used on spinal implants that may be subject to cyclic loading.

It is understood that all spatial references, such as “top,” “inner,” “outer,” “bottom,” “left,” “right,” “anterior,” “posterior,” “superior,” “inferior,” “medial,” “lateral,” “upper,” and “lower” are for illustrative purposes only and can be varied within the scope of the disclosure.

While embodiments of the invention have been illustrated and described in detail in the disclosure, the disclosure is to be considered as illustrative and not restrictive in character. All changes and modifications that come within the spirit of the invention are to be considered within the scope of the disclosure.

We claim:

1. A method comprising:
introducing a residual compressive stress into a bone-engaging portion of an implantable device configured for implantation in a body, the bone engaging portion including a bone-engaging surface; and
texturing the bone-engaging surface of the implantable device to increase a roughness of the bone-engaging surface.

2. The method of claim 1, wherein the introducing a residual compressive stress is performed until the compressive stress is at a first depth in the bone engaging portion, and wherein texturing the bone-engaging surface is performed until the texturing is at a second depth in the implantable device, and wherein the second depth is less than the first depth.

3. The method of claim 1, wherein the introducing a residual compressive stress comprises work-hardening the bone engaging portion of the implantable device.

4. The method of claim 3, wherein the work-hardening includes peening the bone engaging surface of the implantable device.

5. The method of claim 4, wherein the peening is a first shot peening using a first media having a first size, and wherein the work-hardening further comprises a second shot peening using a second media having a second size, the second size being smaller than the first size.

6. The method of claim 3, wherein the work-hardening includes one of:
a forging process; a pressurization process; a water jet process; a drawing process; cold rolling; drawing; deep drawing; pressing; bending; cold forging; cold extrusion; hammering; shearing; and peening.

7. The method of claim 1, wherein the introducing a residual compressive stress in accomplished while the bone-engaging surface is maintained below its melting point.

8. The method of claim 1, wherein the texturing comprises grit blasting the bone-engaging surface of the implantable device.

9. The method of claim 1, wherein the texturing is accomplished by one of: chemical etching; electrical etching; sanding; electrical discharge; machining; grit-blasting; abrading; plasma etching; grit-blasting; abrading; plasma etching; and embedding particles.

10. The method of claim 1, wherein the second depth is less than about 50% of the first depth.

11. A method of treating a bone-engaging portion of an implantable device to maintain fatigue resistance properties while providing surface texturing, the method comprising:
peening the bone-engaging portion of the implantable device to introduce a residual compressive stress into the bone engaging feature, the residual compressive stress having a first depth; and
texturing the peened bone engaging portion to increase a surface roughness of the bone engaging portion, the texturing having a second depth.

12. The method of claim 11, wherein the second depth is less than the first depth.

13. The method of claim 12, wherein the texturing comprises grit blasting the bone-engaging portion of the implantable device.

14. The method of claim 12, further comprising the step of analyzing the bone-engaging portion to determine the desired first depth of the residual compressive stress.

15. The method of claim 14, further comprising the step of correlating the curvature of a verification strip to the desired first depth of the residual compressive stress via peening the verification strip.

16. The method of claim 11, comprising masking a portion of the implantable device prior to the peening the bone-engaging portion.

17. The method of claim 11, wherein the implantable device is a bone screw and the bone-engaging portion includes a threaded surface portion of the bone screw.

18. The method of claim 11, wherein the texturing the peened bone engaging portion comprises machining the bone-engaging portion.

19. An implantable device, comprising:
a bone-engaging portion having a bone-engaging surface and a thickness, wherein the bone-engaging portion has a residual compressive stress extending to a first depth, and
wherein the bone-engaging surface has a roughened texture, the roughened texture penetrating the bone engaging surface to a second depth, the second depth being less than the first depth.

20. The implantable device of claim 19, wherein only a portion of the bone-engaging surface is textured.

21. The implantable device of claim 19, wherein the bone-engaging portion is a threaded portion of a bone screw.

22. The implantable device of claim 19, wherein the bone engaging portion is one of: an outer portion of: an implantable artificial disc; a facet joint replacement implant; an intervertebral spacer; bone plate; an interspinous spacer; a bone screw; a bone anchor; a bone fastener; a fenestrated screw; a corpectomy device; an intramedulary rod; a hip joint replacement implant; a bone pin or rod; a knee joint replacement implant; a shoulder joint replacement implant; an elbow joint replacement implant; a wrist joint replacement implant; an ankle joint replacement implant; a finger joint replacement implant; a toe joint replacement implant; a dental implant; and a maxillofacial/cranial implant.
